(19) United States
(12) Patent Application Publication
(10) Pub. No.: US 2022/0079485 A1
(43) Pub. Date: Mar. 17, 2022

(54) SYSTEMS AND METHODS FOR REMOTE ASSESSMENT USING GAZE TRACKING

(71) Applicant: THE REGENTS OF THE UNIVERSITY OF CALIFORNIA, Oakland, CA (US)

(72) Inventors: Leanne Chukoskie, La Jolla, CA (US); Joseph Snider, La Jolla, CA (US); Phyllis Townsend, La Jolla, CA (US)

(21) Appl. No.: 17/419,913

(22) PCT Filed: Dec. 31, 2019
(86) PCT No.: PCT/US2019/069109

§ 371 Date: Jun. 30, 2021

Related U.S. Application Data
(60) Provisional application No. 62/787,109, filed on Dec. 31, 2018.

(51) Int. Cl. A61B 5/16; A61B 5/11

(52) U.S. Cl. CPC A61B 5/168; A61B 2503/06; A61B 5/1128; A61B 5/163

(57) ABSTRACT

A method of improving one of attention, ASD, and ADHD is disclosed. The method may include presenting first content to a user. The first content may include a first instruction and a first cue. The first instruction may include information for the user to interact with the first cue. Following the first set of instructions may do at least one of the following: improve attention, diagnose ASD, and diagnose ADHD in the user. The method may include tracking first user movement during presentation of the first content. The method may include evaluating the first user movement based on the first instruction to, at least one of, diagnose a first attention level, diagnose ASD, and diagnose ADHD in the user.

### CROSS REFERENCE TO RELATED APPLICATIONS

The present application is a U.S. national phase of PCT International Patent Application No. PCT/US2019/069109, filed Dec. 31, 2019 and titled “SYSTEMS AND METHODS FOR REMOTE ASSESSMENT USING GAZE TRACKING”, which claims priority to U.S. Provisional Application No. 62/787,109, filed Dec. 31, 2018 and titled “SYSTEMS AND METHODS FOR REMOTE ASSESSMENT USING GAZE TRACKING,” the contents of which are incorporated herein by reference in their entirety.

### TECHNICAL FIELD

Various embodiments generally relate to remote assessment. More particularly, various embodiments are related to remote assessment using gaze tracking games.

### BRIEF SUMMARY OF EMBODIMENTS

Embodiments of the disclosure may be directed to a method of improving attention. The method may include presenting first content to a user. The first content may be presented via a display. The first content may include a first instruction and a first cue. The first instruction may include information for the user to interact with the first cue. Following the first set of instructions may improve attention in the user. The method may include tracking first user movement during presentation of the first content. The first user movement may be tracked with a movement tracking device. The method may include evaluating the first user movement based on the first instruction to diagnose a first attention level in the user. Evaluation may be performed with a physical computer processor.

In embodiments, the method may further include presenting second content to the user. The second content may be presented via the display. The second content may be dynamically adjusted based on an evaluation of the first user movement. The second content may include a second instruction and a second cue. The method may include tracking second user movement during presentation of the second content. The second user movement may be tracked with the movement tracking device. The method may include evaluating the second user movement based on the second instruction to diagnose a second attention level in the user.

In embodiments, the first user movement may include movement of one of a user's eyes and a head.

In embodiments, the first instruction may include one of an instruction to focus the first user movement on the first cue, to focus the user movement away from the first cue, to focus the user movement on an opposite side of the first cue, and to use the first cue to direct future user movement. It should be appreciated that the first instruction may include more specific instructions (e.g., look at a ghost, do not look at a turtle, look at a fish, etc.).

In embodiments, the first cue may dynamically respond to the first user movement. For example, the first cue may disappear when the first user movement comes within the hit box of the first cue. This may also start a tally, or add to an existing one, of the number of cues that have been successfully “captured.” As will be described herein, the accuracy and timing of the first user movement may also be tracked. It should be appreciated that the first cue may otherwise dynamically respond to the first user movement (e.g., cause a sound to be played, cause the first cue to move, cause the first cue to change size, etc.)

In embodiments, tracking the first user movement may include tracking an accuracy of the first user movement in relation to the first cue and tracking a timing of the first user movement in relation to the first cue.

In embodiments, the first cue may be presented for 50 milliseconds.

Some embodiments of the disclosure may also be directed to a method of improving autism spectrum disorder (ASD). The method may include presenting first content to a user. The first content may be presented via a display. The first content may include a first instruction and a first cue. The first instruction may include information for the user to interact with the first cue. Following the first set of instructions may improve symptoms of ASD in the user. The method may include tracking first user movement during presentation of the first content. The first user movement may be tracked with a movement tracking device. The method may include evaluating the first user movement based on the first instruction to diagnose ASD in the user. Evaluation may be performed with a physical computer processor.

In embodiments, the method may further include presenting second content to the user. The second content may be presented via the display. The second content may be dynamically adjusted based on an evaluation of the first user movement. The second content may include a second instruction and a second cue. The method may include tracking second user movement during presentation of the second content. The second user movement may be tracked with the movement tracking device. The method may include evaluating the second user movement based on the second instruction to determine changes to symptoms of the ASD in the user.

In embodiments, the first user movement may include movement of one of a user's eyes and a head.

In embodiments, the first instruction may include one of an instruction to focus on the first cue, look away from the first cue, look at an opposite side of the first cue, and use the first cue to direct future movement.

In embodiments, the first cue may dynamically respond to the first user movement by disappearing.

In embodiments, tracking the first user movement may include tracking an accuracy of the first user movement and tracking a timing of the first user movement.

In embodiments, the first cue may dynamically respond to the first user movement based on the first user movement being within less than a 2-inch by 2-inch square from a perspective of the user.

Embodiments of the disclosure may be directed to a method of improving attention deficit hyperactivity disorder (ADHD). The method may include presenting first content to a user. The first content may be presented via a display. The first content may include a first instruction and a first cue. The first instruction may include information for the user to interact with the first cue. Following the first set of instructions may improve symptoms of ADHD in the user. The method may include tracking first user movement during presentation of the first content. The first user movement may be tracked with a movement tracking device. The method may include evaluating the first user movement based on the first instruction to diagnose ADHD in the user. Evaluation may be performed with a physical computer processor.

In embodiments, the method may further include presenting second content to the user. The second content may be presented via the display. The second content may be dynamically adjusted based on an evaluation of the first user movement. The second content may include a second instruction and a second cue. The method may include tracking second user movement during presentation of the second content. The second user movement may be tracked with the movement tracking device. The method may include evaluating the second user movement based on the second instruction to determine changes to symptoms of the ADHD in the user.

In embodiments, the first user movement may include movement of one of a user's eyes and a head.

In embodiments, the first instruction may include one of an instruction to focus on the first cue, look away from the first cue, look at an opposite side of the first cue, and use the first cue to direct future movement.

In embodiments, the first cue may dynamically respond to the first user movement by providing audio.

In embodiments, tracking the first user movement may include tracking an accuracy of the first user movement and tracking a timing of the first user movement.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates an example environment in which embodiments of the disclosure may be implemented.

FIG. 2A illustrates a user playing an example game, in accordance with various embodiments of the present disclosure.

FIG. 2B illustrates a plot of changes to a user from playing the game.

FIG. 3 illustrates example graphs relating to attention, in accordance with various embodiments of the present disclosure.

FIG. 4A illustrates an example game, in accordance with one embodiment of the present disclosure.

FIG. 4B illustrates an example game, in accordance with one embodiment of the present disclosure.

FIG. 5A illustrates an example game, in accordance with one embodiment of the present disclosure.

FIG. 5B illustrates an example game, in accordance with one embodiment of the present disclosure.

FIG. 5C illustrates an example game, in accordance with one embodiment of the present disclosure.

FIG. 5D illustrates an example game, in accordance with one embodiment of the present disclosure.

FIG. 5E illustrates an example game, in accordance with one embodiment of the present disclosure.

FIG. 5F illustrates an example game, in accordance with one embodiment of the present disclosure.

FIG. 5G illustrates an example game, in accordance with one embodiment of the present disclosure.

FIG. 5H illustrates an example game, in accordance with one embodiment of the present disclosure.

FIG. 5I illustrates an example game, in accordance with one embodiment of the present disclosure.

FIG. 5J illustrates an example game, in accordance with one embodiment of the present disclosure.

FIG. 6A illustrates an example game, in accordance with one embodiment of the present disclosure.

FIG. 6B illustrates an example game, in accordance with one embodiment of the present disclosure.

FIG. 6C illustrates an example game, in accordance with one embodiment of the present disclosure.

FIG. 6D illustrates an example game, in accordance with one embodiment of the present disclosure.

FIG. 6E illustrates an example game, in accordance with one embodiment of the present disclosure.

FIG. 7A illustrates an example game, in accordance with one embodiment of the present disclosure.

FIG. 7B illustrates an example game, in accordance with one embodiment of the present disclosure.

FIG. 7C illustrates an example game, in accordance with one embodiment of the present disclosure.

FIG. 7D illustrates an example game, in accordance with one embodiment of the present disclosure.

FIG. 7E illustrates an example game, in accordance with one embodiment of the present disclosure.

FIG. 7F illustrates an example game, in accordance with one embodiment of the present disclosure.

FIG. 7G illustrates an example game, in accordance with one embodiment of the present disclosure.

FIG. 8 illustrates example games, in accordance with one embodiment of the present disclosure.

FIG. 9 illustrates an example computing component that may be used to implement features of various embodiments of the disclosure.

FIG. 10 is a flow chart illustrating example operations that can be performed to improve attention and symptoms of conditions in accordance with various embodiments of the present disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Approximately 11% of school age children have attention deficit, hyperactivity disorder (ADHD), which is characterized by specific difficulties in control of attentional focus. In addition, about 2.5% of children have autism spectrum disorder (ASD) and the majority of these have specific deficits in attention orienting. ADHD symptoms are also co-morbid in as many as half of ASD children.

Attention is an underappreciated cognitive skill important to succeed in education. Students need to be able to orient their attention to the material of interest, focus their attention on the material for a sufficient amount of time, and inhibit any distractions that may pull their attention away from that focus. Common examples of attention issues include failure to orient to the board and the teacher, lack of focus on texts the student should be reading, and a failure to inhibit distractions when working through a math problem. Attention skills are trainable, and school administrators are seeking tools to enable their teachers and therapists to train these skills. Traditional tools to evaluate and train these skills require subjective evaluation by a medical professional in a lab setting. These traditional techniques cannot scale to cover multiple people simultaneously across the world because there are not enough medical professionals to evaluate and diagnose people, they require lab settings, they are not spread out enough geographically, and they do not support remote assessment of users. For example, eye movements, which are readily quantifiable, may be used as a measure of attention. Eye movements may be used because the neural systems that control eye movements are shared with the systems that control attention; in other words, attention and eyes move together

The presently disclosed technology may capitalize on the inherent link between gaze and attention by improving attention skills of challenged students through concrete observations of their gaze performance and gaze data. These improvements in attention skill may increase academic success, and as such have far-reaching, cumulative effects. The presently disclosed technology may leverage the collected gaze data to evaluate, diagnose, train, and improve attention, symptoms of ADHD, symptoms of ASD, and other conditions remotely and can be scaled for larger populations, which could not be done with traditional tools.

For example, the tracked gaze data may be evaluated remotely by various reviewers to determine accuracy of the user's gaze, stability of the user's gaze, speed of the user's gaze, precision of the user's gaze, and other elements of the user's gaze to evaluate changes to the user's gaze over time and determine whether a user may have attention issues, ADHD, ASD, and other conditions. The presently disclosed technology may include tracking user movement during presentation of content to a user to evaluate the user's attention. For example, the content may include a suite of gaze-driven video games to measure, train, and track attention skills including orienting, focus, and inhibitory control. For example, some of these games, as will be discussed in greater detail below, harness fast eye movements used to reorient gaze about four times per second. It should be appreciated that in some embodiments, other content, such as videos, pictures, and other content may be presented to a user.

The presently disclosed technology may include games that may measure and build attention skills by demanding the timing and accuracy to orient and control gaze shifts. In embodiments, inhibitory control may be trained, by making subtle changes that turn a target into a distractor to be avoided. The games may be played with, for example, an eye-tracking sensor such that inattention or off-task behavior leads to in-game failures, which can be used to measure and shape attention control. The games may include one or more of one or more cues, one or more videos, one or more graphics, one or more instructions, one or more user interfaces, and other features. Traditional games may cause a user to look at a screen for recreation and entertainment. Some of these traditional games may evaluate body control and body movement. However, these traditional games do not evaluate ASD, ADHD, or other conditions. These traditional games are also not directed to improve attention, symptoms of ADHD, symptoms of ASD, and other conditions. Embodiments of the presently disclosed technology can be used to track user movement, evaluate users with at least one of ASD, ADHD, or other conditions based on the user movement, and improve symptoms of these conditions, and in some embodiments, the conditions themselves.

The content presented to the user may include one or more cues, one or more instructions, one or more graphics, one or more audio accompaniments, and other audio-visual accompaniments. Cues may include dynamic interactable objects presented to the user. Interacting with the cues may cause the cues to dynamically respond by causing the cue to disappear, causing the cue to move, causing a sound to be played, causing another cue to appear, causing a graphic to appear, and other dynamic responses. Cues may be presented as graphics on a screen that the user can interact with. As discussed in greater detail below, some examples of cues may include a ghost a user needs to focus user movement on to capture, a turtle the user needs to avoid through user movement, a direction a tiger needs to follow to reach the next part of a path. It should be appreciated that cues can take varied embodiments depending on the content. It should also be appreciated that cues can change from content to content. For example, cues may become faster or slower from first content to second content, cues may disappear more quickly or more slowly, cues may have smaller or larger hitboxes, cues may be simultaneous, more cues may be presented, cues may become smaller, larger, or more varied, cues may have longer or shorter fixation times for the cue to dynamically respond, and other changes.

The instructions may include directions for the user to interact with the content presented. Following the instructions may improve attention issues, symptoms of ADHD, symptoms of ASD, and other conditions. Example instructions are presented in further detail below.

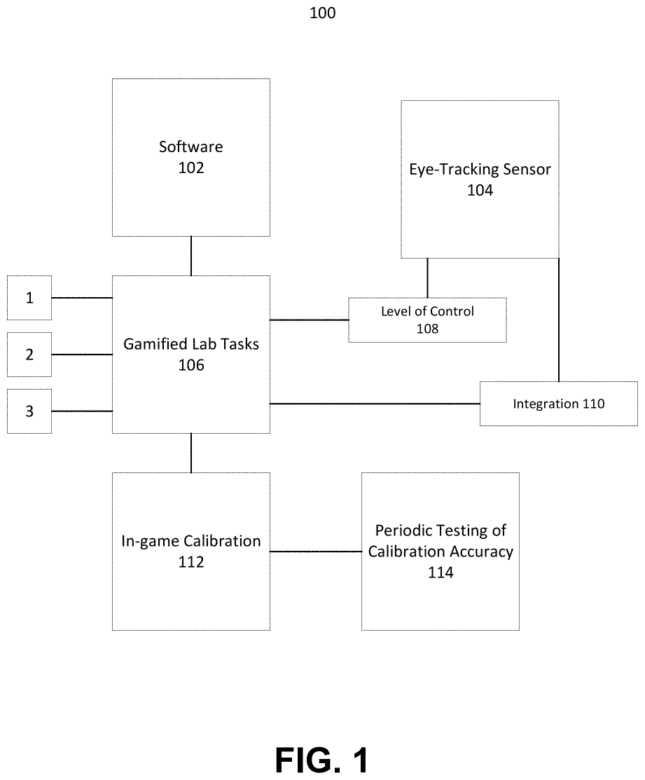

For example, FIG. 1 illustrates an example environment 100 in which embodiments of the disclosure may be implemented. As illustrated, the laboratory tasks, 1, 2, and 3, may be turned into gamified lab tasks 106. Gamified lab tasks 106 may be played using eye-tracking sensor 104. Gamified lab tasks 106 may be subject to in-game calibration 112 to improve data collection and the eye-tracking sensor 104. In-game calibration 112 may include looking at corners of a display, specific cues on the display, and other directions to calibrate eye-tracking sensor 104 with body movement. It should be appreciated that other calibration techniques may be used. For example, scanning through an array of cues, fixating on a single cue, and so on.

Eye-tracking sensor 104 may include one or more optical imaging systems, such as one or more cameras, one or more infrared sensors, and other optical imaging systems. Eye tracking sensor 104 may be mounted to a pair of glasses to be worn by a user, mounted to a display (e.g., a screen, a head-mounted display, and the like) the content will be presented on, integrated with the display or the pair of glasses, or otherwise provided to a user. Eye-tracking sensor 104 may generate gaze data based on capturing the user's eyes over a period of time. The gaze data may include data from the content presented to the user such that the gaze data informs accuracy, precision, timing, and other elements of the user's gaze. The gaze data may be used to determine a severity of attention issues, ADHD, ASD, and other conditions.

In embodiments, calibration 112 may be periodically checked 114 to ensure calibration 112 is within a threshold range. Periodic calibration 114 may be based on time (e.g., every 30 seconds, every minute, every 5 minutes, and so on), based on game events (e.g., after user input in response to a cue, after the end of a game, and the like), and other parameters. The threshold range may be within 10% of a hitbox around a cue (e.g., a region around a cue that may still activate the cue). The hitbox may be five percent of a size of a cue, ten percent of a size of a cue, 25% of a size of a cue, and so on. The hitbox size may change between content presented.

The presently disclosed technology may include software 102 of gamified lab tasks 106 that may be reliable, sensitive, specific, playable and may be able to collect a large volume of data. The presently disclosed technology may use in-game assessments that measure and evaluate performance changes. For example, gamified versions of laboratory tasks 106 may be controlled 108 by a user using an eye-tracking system or a movement tracking device. The content may be presented on a display, AR system, VR system, and other environments. Gaze may be used to control 108 the in-game assessments. The eye-tracking system may include eye tracking sensor 104. Eye-tracking sensor 104 may be calibrated 112 to games suite 106, and otherwise integrated 110 with gamified lab tasks 106. Factors such as calibration 112 and headbox size (i.e., trackable volume) in eye-tracking sensor 104 may improve the presently disclosed technology.

In embodiments, camera-based eye tracking may use image processing algorithms to localize the position of the pupil relative to the eye socket to determine gaze position. For example, some image processing algorithms may take an image of a user's eyes and extract different features of the eye from the image. The features may include a shape of the eye, light refraction from a part of the eye, reflection of different parts of the eye, a shape of the iris, a shape of the cornea, positions on the eye, and other features. Relevant factors that affect eye tracking may include camera resolution, ambient lighting tolerance, sample rates, and algorithmic robustness. The eye-tracking sensor may include an onboard chip which reduces the load required of the computing system to which the tracker is connected. These trackers may have a larger headbox region of space in front of the tracker in which gaze position can be reliably detected.

While an eye tracking system is described with respect to FIG. 1, it should be appreciated that a movement tracking device may be used instead. The motion tracking device may include an eye tracking system, as well as a head tracking system, a shoulder tracking system, and other tracking systems that track of other body parts that may affect a user's gaze. The movement tracking device may use one or more cameras, one or more motion sensors, one or more infrared sensors, one or more gyroscopes, and other sensors to capture movement of the eyes, head, shoulder, and other body parts that may affect where a user is looking. The movement tracking device may use the captured movement to determine where a user is looking. The additional data gathered from the motion tracking device may be used to improve accuracy of the gaze data.

The gaze data collected may be used for various assessment purposes. For example, at the administrator level, an interactive tool may be generated that shows overall usage frequency by date ranges, as well as general overviews of performance.

Within the classroom or study group at the teacher level, the UI/UX may focus on interfaces that show performance by individual child, including usage frequency and dates performance of each child as compared to average peer performance.

The individual children who play the games may have access to their own progress through the games and assessments that encourage them to keep playing. The UI/UX experience may be adapted to children as well as creating in-game incentives for them to complete the attention assessment tasks that are separate from the games.

The presently disclosed technology may be used to assess orienting, focus, and inhibitory control. The presently disclosed technology may use, for example, the Unity game development platform to implement the suite of games. It should be appreciated that other game development platforms may be used to implement the presently disclosed technology, including Godot, Torque, libGDX, Source, Blender Game Engine, Unreal Engine 4, CryEngine, Unigine, Construct, OpenGL, and other game engines. Data management may use a software interface that may be able to connect to a database. Playable and tested versions of the attention games may be validated using data recording. The presently disclosed technology may include a system that rewards compliance with extra in-game items and achievements when individuals complete each assessment.

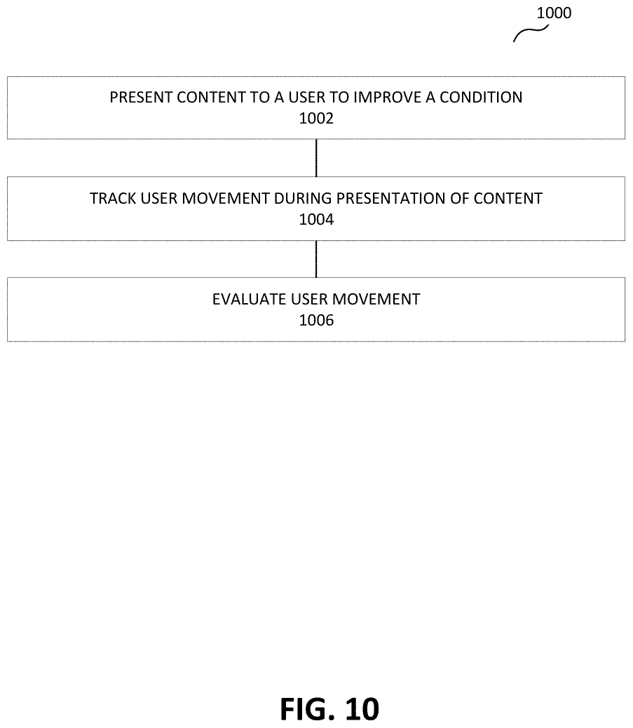

FIG. 10 is flow chart 1000 illustrating example operations that can be performed to improve attention and symptoms of conditions in accordance with various embodiments of the present disclosure. The operations of the various methods described herein are not necessarily limited to the order described or shown in the figures, and it will be appreciated, upon studying the present disclosure, variations of the order of the operations described herein that are within the spirit and scope of the disclosure.

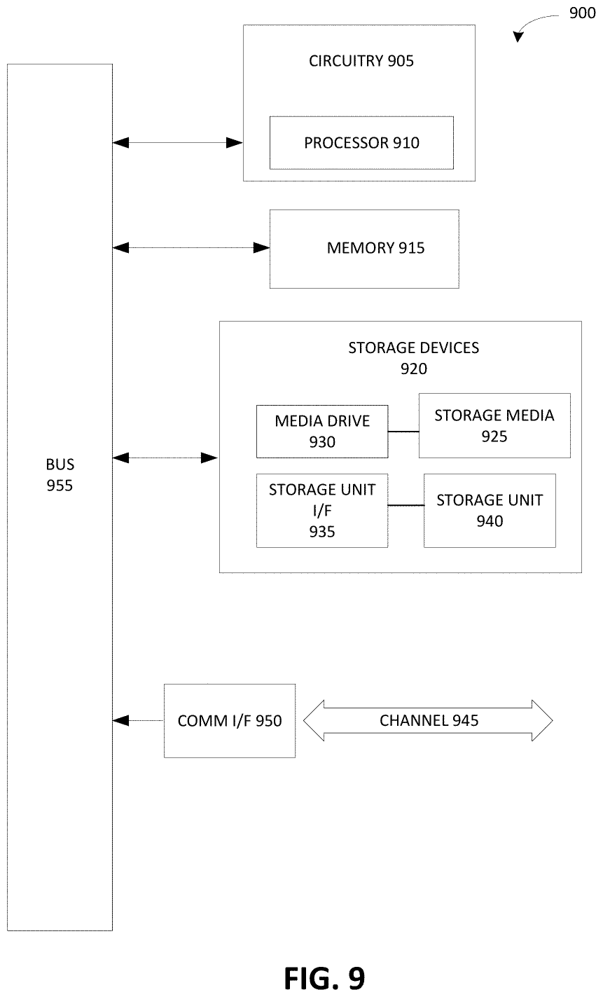

The operations and sub-operations of flow chart 1000 may be carried out, in some cases, by one or more of the components, elements, devices, and circuitry of environment 100 and/or computing component 900, described herein and referenced with respect to at least FIGS. 1 and 9, as well as sub-components, elements, devices, and circuitry depicted therein and/or described with respect thereto. In such instances, the description of flow chart 1000 may refer to a corresponding component, element, etc., but regardless of whether an explicit reference is made, it will be appreciated, upon studying the present disclosure, when the corresponding component, element, etc. may be used. Further, it will be appreciated that such references do not necessarily limit the described methods to the particular component, element, etc. referred to. Thus, it will be appreciated that aspects and features described above in connection with (sub-) components, elements, devices, circuitry, etc., including variations thereof, may be applied to the various operations described in connection with methods 1000 without departing from the scope of the present disclosure.

At operation 1002, flow chart 1000 includes presenting content to a user to improve a condition. A user may select one of the available content. The selected content may be presented. The content may include instructions for the user to follow. The instructions may indicate how to interact with cues that are to be presented in the content. The instructions can provide directions for the user to focus on a cue for one or more milliseconds, or seconds; directions on how to tell when a cue may appear based on graphics, cues, audio, and other audio-visual accompaniments; and other directions as described herein.

An interactive portion of the content may follow the instructions. The user may start the interactive portion of the content by focusing user movement on a cue in the content. Cues, graphics, audio, and other audio-visual accompaniments may be presented during the interactive portion of the content. Based on the user movement, the content may dynamically respond to the user movement. For example, the cue may disappear, the cue may move, additional cues may be presented, graphics may be presented, audio may be presented, and other audio-visual accompaniments may be presented.

A results section may follow the interactive portion of the content. The results section may include a number of successful interactions, a number of missed interactions, the quality of all interactions, and other information. In-game rewards may be provided to the user to improve user interaction and retention.

At operation 1004, flow chart 1000 includes tracking user movement during presentation of the content. The user movement may include pupil movement, eye movement, cornea movement, iris movement, head movement, shoulder movement, and movement of other body parts that may affect a user's gaze. The user movement may be tracked during an interactive portion of the content, as described above. The user movement may be used to generate gaze data specifying gaze as a function of time during the interactive portion of the content. The gaze data may include data gathered from the content such that the gaze data can be used to identify elements of a user's attention (e.g., accuracy, precision, steadiness, timing, and the like).

At operation 1006, flow chart 1000 includes evaluating user movement. The user movement may be evaluated based on the content presented. For example, the gaze data may be used to identify attention issues in the user, whether the user has ADHD, whether the user has ASD, and whether the user has other conditions. Following the instructions may improve attention in the user, symptoms of ADHD in the user, symptoms of ASD in the user, and other conditions.

As will be appreciated, flow chart 1000 may be repeated for additional content to be presented to the user. The additional content may be dynamically adjusted based on an evaluation of the user movement during the first content. For example, if the user had mostly successful interactions, the additional content may be more difficult (i.e., smaller hitboxes, faster cues, cues that disappear more quickly, and the like). If the user had mostly missed interactions, the additional content may be easier (i.e., larger hitboxes, slower cues, cues that disappear slower, and the like). The quality of the interaction (e.g., stability, accuracy, and the like) may affect the evaluation.

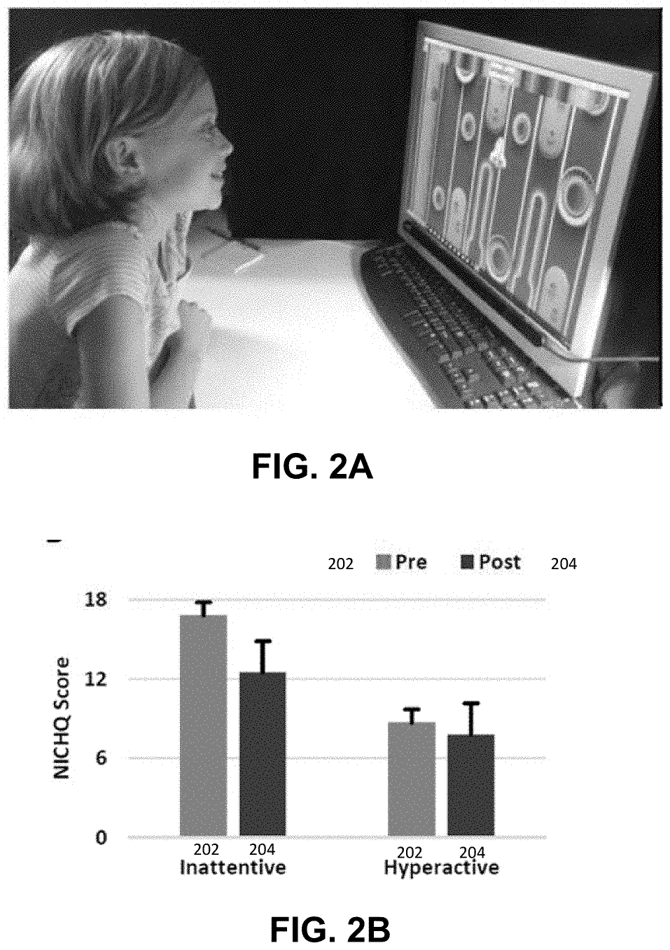

The presently disclosed technology may quantify when users are engaged or disengaged. FIG. 2A illustrates a user playing an example game, in accordance with various embodiments of the present disclosure. Multiple anecdotal observations may suggest that these games might measure or train attention in a broader manner and in a way that could have impact on real-life skills (see FIG. 2B). The effects of training on related tasks may represent “near transfer,” but effects on less related tasks may be examples of “far transfer.” The suite of training games presently disclosed can elicit far transfer. The presently disclosed technology discloses a system with built-in feedback that can be used independently in schools by a wide range of children.

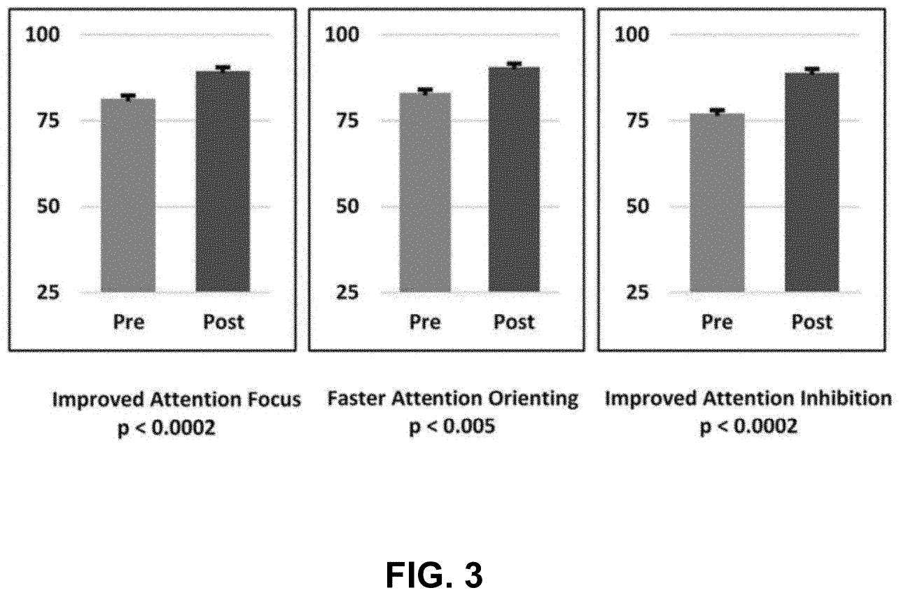

In addition to qualitative reports of attention improvements, post-training improvements were found in attention orienting, attention, and gaze inhibition (see FIGS. 2B and 3).

The presently disclosed technology may include a backend data management tool that may be automated and connected with a database ensuring data security and reliability. In embodiments, the data management tool may produce useful summary plots at different levels of analysis (e.g., student, groups of students, larger cohort, etc.).

Using the presently disclosed technology, improved attention focus, faster attention orienting, and improved attention inhibition may be measured. FIG. 3 illustrates example graphs relating to attention, in accordance with various embodiments of the present disclosure. The vertical axes may represent a percent correct. Chance performance may be about 25%. Post-training group tests show improvement in attention focus (i.e., overall performance), attention orienting (i.e., performance with a short time to orient attention), and attention inhibition (i.e., variability in eye movement around a fixation point).

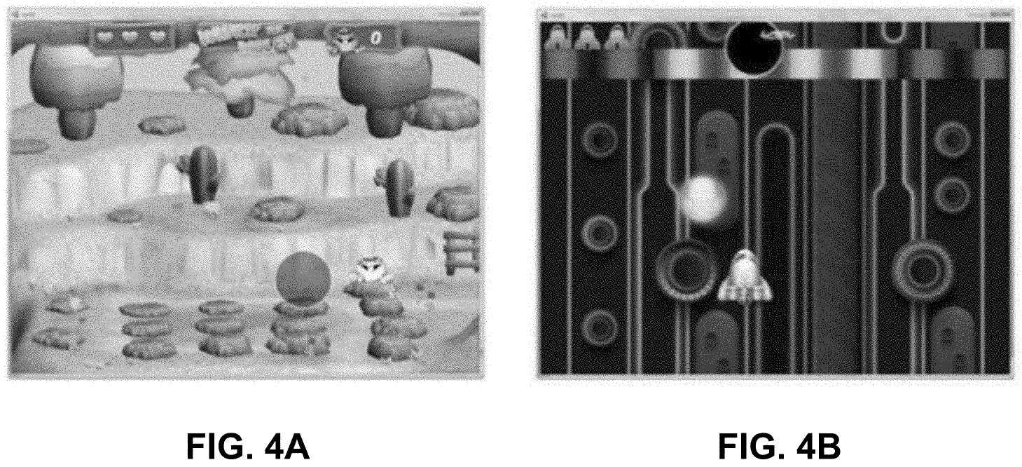

The eye-tracker may be used to play example games (see white circle filled with gray in FIGS. 4A, 4B, 5A, 5B, 5C, 5D, 5E, 5F, 5G, 5H, 5I, 5J, 6A, 6B, 6C, 6D, 6E, 7A, 7B, 7C, 7D, 7E, 7F, 7G, and 8) as a tool to interact with the games. By creating “target windows,” or hitboxes, around targets that appear in the games, gaze data on the accuracy and timing of hitting these targets may be collected, as well as the steadiness of fixation on an object. FIG. 4A may illustrate a whack-a-mole-type game. FIG. 4B may illustrate a space-race-type game.

A suite of behavioral tasks may be used to assess attention in the lab environment. The games may include gamified performance assessments of attention orienting, focus, and inhibition. For example, a combination of tasks may allow measurement of inhibitory control directly and in the context of a higher cognitive load in a task-switching scenario. A pro-saccade task may estimate the timing and accuracy of eye movements made to small characters that appear suddenly on the screen. The characters may be zapped by looking at them as quickly and accurately as possible. After a round of the pro-saccade task, players may be instructed to shift a gaze to inhibit looks to the target about to appear. A change in the color of the central fixation point may be used to reinforce that this is an anti-saccade (or “don't look”) trial. The player may be rewarded for looking to the position opposite to the target at an equivalent distance. In embodiments, the two types of trials may be mixed in subsequent rounds. The change in color of the fixation cue may denote an upcoming “don't look” trial. Accuracy and timing may be measured in both single condition blocks, as well as the mixed block.

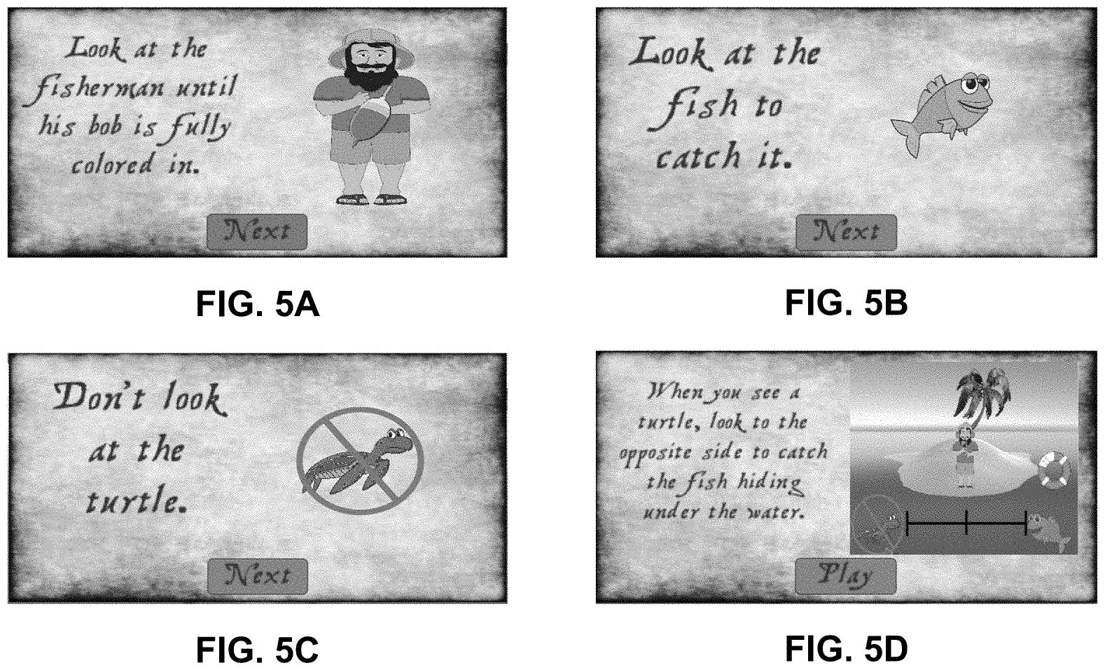

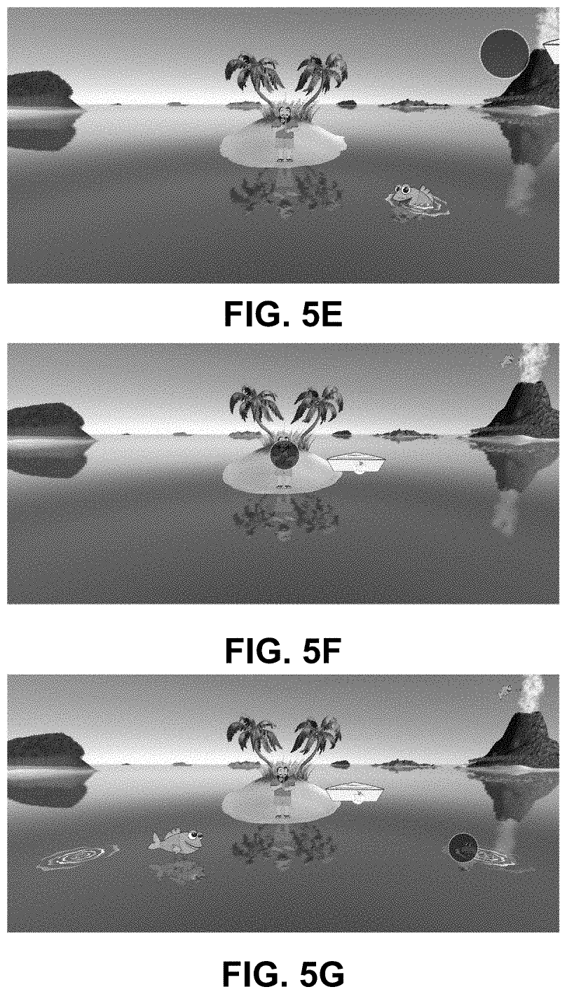

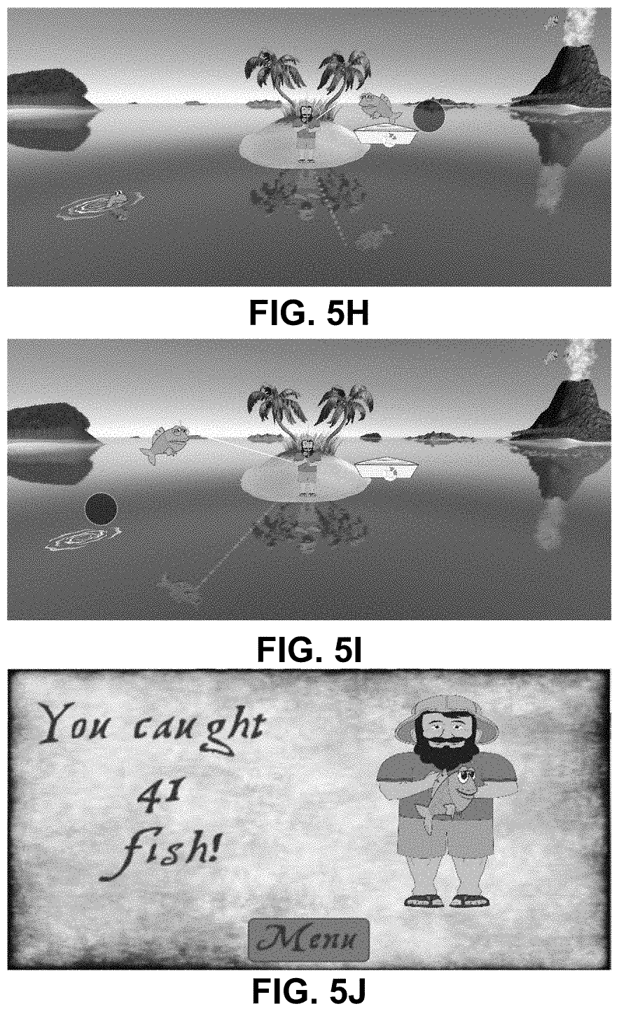

In embodiments, as illustrated in FIGS. 5A, 5B, 5C, 5D, 5E, 5F, 5G, 5H, 5I, and 5J, a lake and serene music may set the scene. After a series of instruction screens (see FIGS. 5A, 5B, 5C, and 5D), a fisherman holding a pole may appear in the center of the screen. The instructions may indicate looking at the fisherman for a period of time to start the game, looking at a fish to catch the fish, not looking at a turtle, and, when a turtle appears on the screen, looking on the opposite side to catch a fish. The player may control the tip of the fishing line with the player's gaze (e.g., white circle filled with gray; see top right corner of FIG. 5E). For example, the game may start by keeping a steady gaze on the fisherman (see FIG. 5F). Ripples may appear in one of two fixed locations, such as, for example, to the left and right of screen center, followed by an animal. The player's task may be to catch the fish (see FIG. 5I) but avoid the turtles (see FIG. 5H). The player's goal may be to catch this fish by moving his gaze to a spot above the ripple. When successful, the fish may be caught on the line and brought back to the fisherman (see FIG. 5I). If the player misses the fish, the fish may land back in the water, thereby missing an interaction. In embodiments, the player's gaze may return to the fisherman to launch the next trial. On some of the game events, a turtle may appear after the ripple instead of a fish. In this case, the player may gaze at the opposite target location, where a fish may jump without a ripple. If the player looks at the turtle by mistake, the fish jumps out of the water on the other side and is not caught (see FIG. 5G). Sound effects may be included in the game. For example, sound effects may include the line casting, line reeling in, and splashes. After all trials have been presented, a popup screen may tell the player how many fish were caught (see FIG. 5J). Pressing OK may return a player to the main menu.

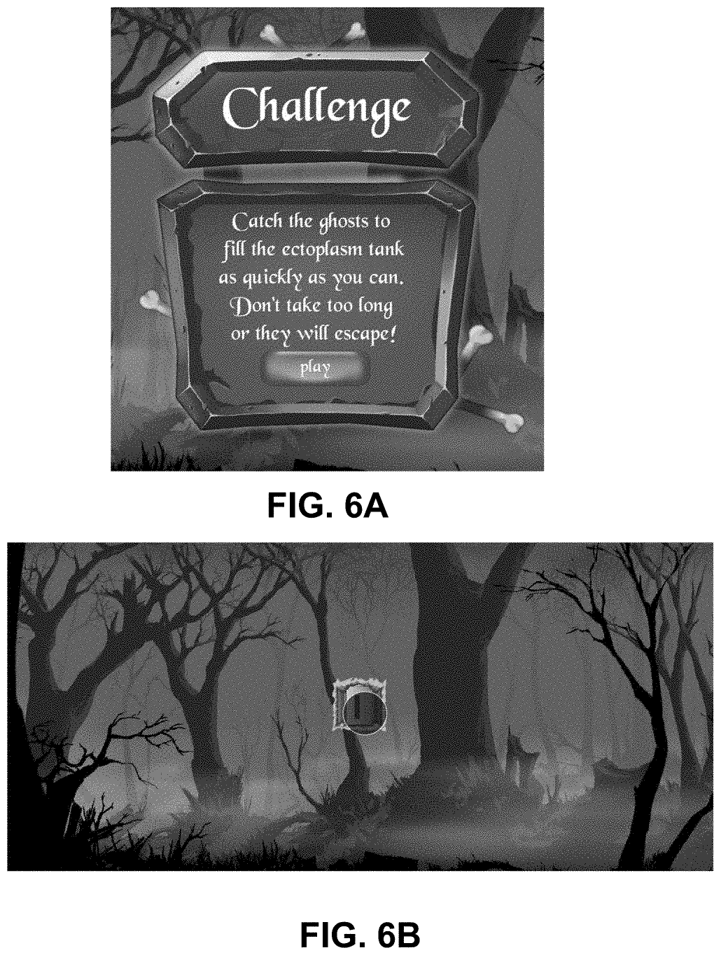

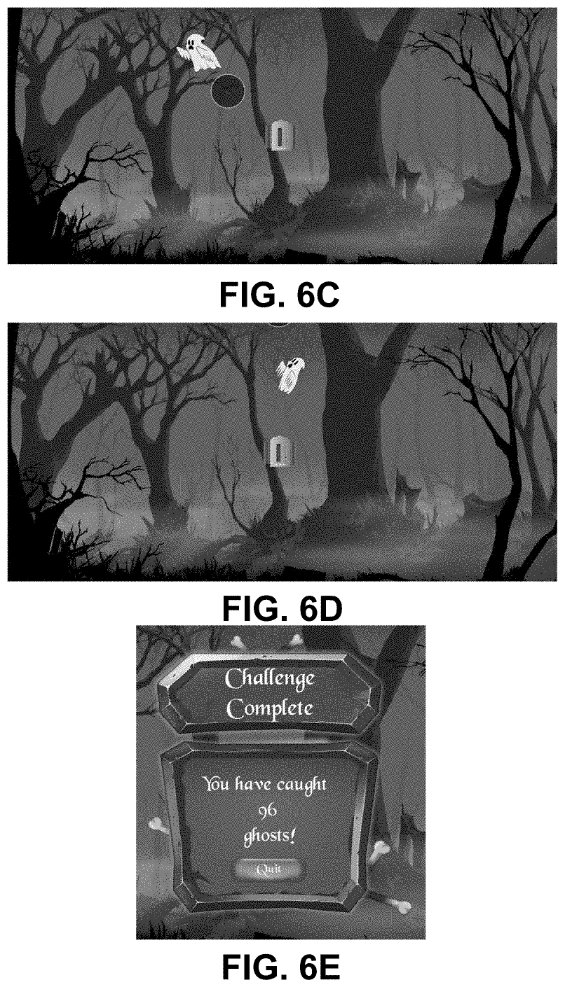

In embodiments, as illustrated in FIGS. 6A, 6B, 6C, 6D, and 6E, a spooky background and spooky music may set the scene (see FIG. 6A). A holding tank may sit at the center of the screen, demanding the player's steady gaze (e.g., white circle) to start the game (see FIG. 6B). Ghosts may appear one at a time at locations located in a circle surrounding the container (see FIG. 6C). In embodiments, multiple ghosts may appear simultaneously. The player's gaze may return to the center container before the next ghost may be shown. When the player's gaze hits the ghost before the timeout, the ghost may be sucked into the container, accompanied by an amusing “scream”, and the progress bar on the container may increase (see FIG. 6D). If the player misses, the screen may briefly fade out. After all ghosts have been presented, a popup screen may tell the player how many ghosts were hit (see FIG. 6D). Pressing quit returns to the main menu (see FIG. 6E).

This task may be used to evaluate gaze focus (i.e., variability in eye movement around a fixation point), as well as behavioral inhibition (i.e., the behavioral manifestation of attention inhibition) in two contexts.

For example, one game may be a based on a Cued Spatial Attention Task (such as the “E-task”). Generally, the task/game may have a central fixation cross on a computer monitor flanked by boxes on the left and right. Trials may begin with an attention directing cue (e.g., one of the boxes brightens). Following a post-cue delay (e.g., short at 100 ms or long at 800 ms), a target (e.g., letter “E” pointing up, down, left or right) is presented in either the cued or the opposite uncued location. In this task, the subject may move a joystick lever to indicate target orientation (e.g., up, down, left, right). The task is controlled for visual perception (e.g., the target may be masked by a feature mask after 50 ms) and motor response (e.g., after the target is masked, participants may have up to 2000 ms to respond). Results from several studies using this task demonstrate slowed disengagement and shifting of attention in ASD children and adults. The Cued Spatial Attention Task may be provided to evaluate attention orienting speed (i.e., accuracy with a short time to orient attention), focus (i.e., overall performance—a measure of attention on-task), and inhibitory control (i.e., efficiency of inhibiting attention to a distracting location).

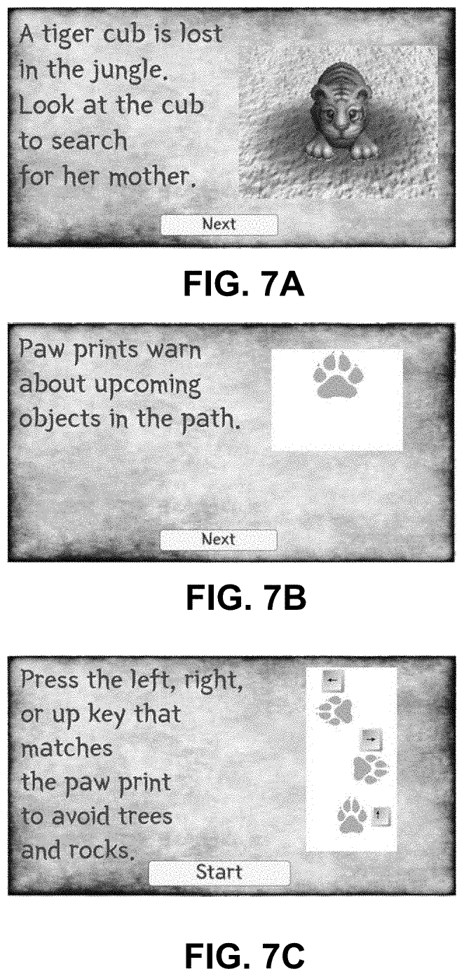

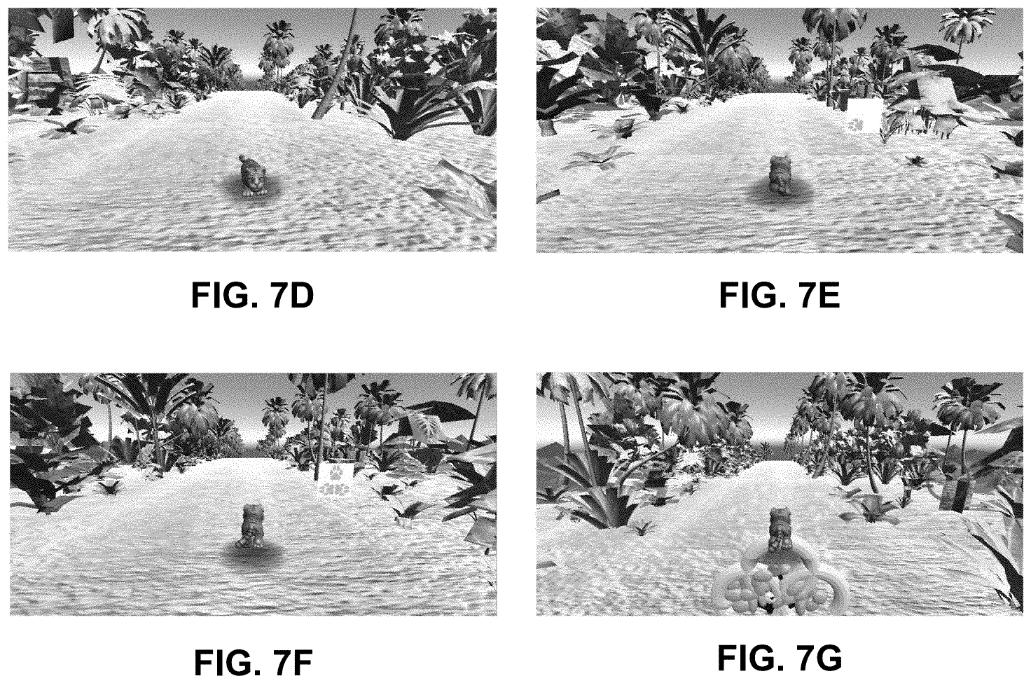

In embodiments of the game, as illustrated in FIGS. 7A, 7B, 7C, 7D, 7E, 7F, and 7G, an endless runner game, a tiger cub may be directed based on peripheral cues to navigate obstacles in its path on the journey back to its mother. The game opens with instructions (see FIGS. 7A, 7B, and 7C). The instructions may provide directions on how to interact with the cues. The cues may have an orientation indicating which direction to choose. The player may look at the tiger cub (e.g., white circle) for it to turn around and begin to run, thereby beginning the game (see FIG. 7D). The player may be presented with cues (see FIGS. 7B, 7C, 7E, and 7F) which are subsequently hidden to control presentation time and tax attention instead of perceptual memory (see FIG. 7F). For example, the cue in FIG. 7E may indicate the correct direction is left. In FIG. 7F, the correct directions may be up, left, and right. The instructions may present the user with directions on how to manipulate the tiger by interacting with cues. Interaction based on the right cues may reward the user. The tiger cub may be presented with the obstacle and which ring to jump through based on the briefly presented cues (see FIG. 7G).

This task may be used to evaluate attention orienting speed (i.e., accuracy with a short time to orient attention) and focus (i.e., overall performance—a measure of attention on-task), and inhibitory control (i.e., efficiency of inhibiting attention to a distracting location).

The Attention Network Test (ANT) may use a simple task (i.e., respond to the direction of a central arrow) with orienting cues, alerting cues, and flankers to generate separate scores for the three major attention networks: orienting, alerting, and executive function, including inhibition. The Attention Network Test may measure inhibition of distraction (i.e., accuracy and response time in incongruent flanker conditions).

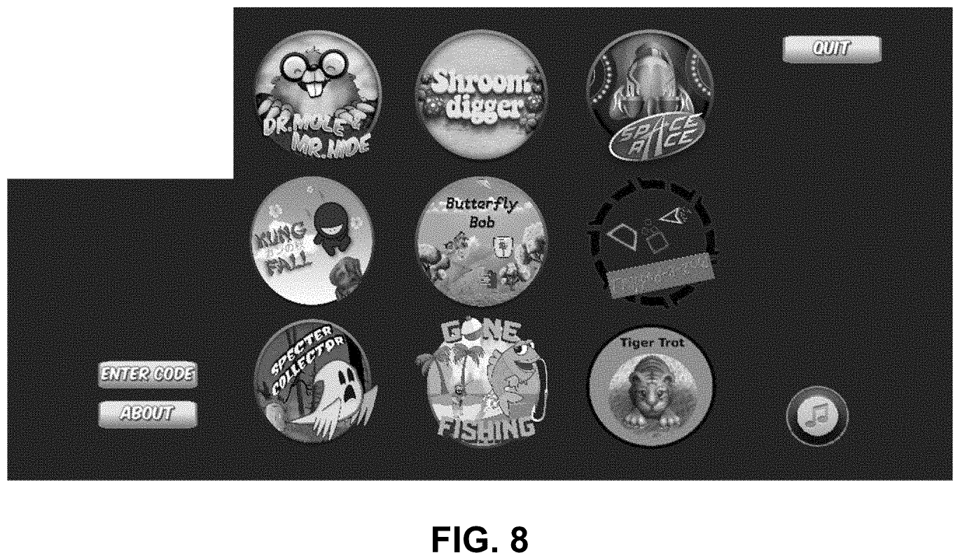

FIG. 8 illustrates example games, in accordance with one embodiment of the present disclosure. Games in addition to those described herein may use an eye tracking sensor to monitor a player's gaze and collect data.

FIG. 9 illustrates example computing component 900, which may in some instances include a processor on a computer system (e.g., control circuit). Computing component 900 may be used to implement various features and/or functionality of embodiments of the systems, devices, and methods disclosed herein. With regard to the above-described embodiments set forth herein in the context of systems, devices, and methods described with reference to FIGS. 1-8 and 10, including embodiments involving the environment 100, one of skill in the art will appreciate additional variations and details regarding the functionality of these embodiments that may be carried out by computing component 900. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. As used herein, a component may be implemented utilizing any form of hardware, software, or a combination thereof. For example, one or more processors, controllers, ASICs, PLAs, PALs, CPLDs, FPGAs, logical components, software routines, or other mechanisms may be implemented to make up a component. In implementation, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components or components of the application are implemented in whole or in part using software, in embodiments, these software elements may be implemented to operate with a computing or processing component capable of carrying out the functionality described with respect thereto. One such example computing component is shown in FIG. 9. Various embodiments are described in terms of example computing component 900. After reading this description, it will become apparent to a person skilled in the relevant art how to implement example configurations described herein using other computing components or architectures.

Referring now to FIG. 9, computing component 900 may represent, for example, computing or processing capabilities found within mainframes, supercomputers, workstations or servers; desktop, laptop, notebook, or tablet computers; hand-held computing devices (e.g., tablets, PDA's, smartphones, cell phones, palmtops, etc.); or the like, depending on the application and/or environment for which computing component 900 is specifically purposed.

Computing component 900 may include, for example, one or more processors, controllers, control components, or other processing devices, such as a processor 910 that may be included in 905. Processor 910 may be implemented using a special-purpose processing engine such as, for example, a microprocessor, controller, or other control logic. In the illustrated example, processor 910 is connected to bus 955 by way of 905, although any communication medium may be used to facilitate interaction with other components of computing component 900 or to communicate externally.

Computing component 900 may also include one or more memory components, simply referred to herein as main memory 915. For example, random access memory (RAM) or other dynamic memory may be used for storing information and instructions to be executed by processor 910 or 905. Main memory 915 may also be used for storing temporary variables or other intermediate information during execution of instructions to be executed by processor 910 or 905. Computing component 900 may likewise include a read only memory (ROM) or other static storage device coupled to bus 955 for storing static information and instructions for processor 910 or 905.

Computing component 900 may also include one or more various forms of information storage devices 920, which may include, for example, media drive 930 and storage unit interface 935. Media drive 930 may include a drive or other mechanism to support fixed or removable storage media 925. For example, a hard disk drive, a floppy disk drive, a magnetic tape drive, an optical disk drive, a CD or DVD drive (R or RW), or other removable or fixed media drive may be provided. Accordingly, removable storage media 925 may include, for example, a hard disk, a floppy disk, magnetic tape, cartridge, optical disk, a CD or DVD, or other fixed or removable medium that is read by, written to or accessed by media drive 930. As these examples illustrate, removable storage media 925 may include a computer usable storage medium having stored therein computer software or data.

In alternative embodiments, information storage devices 920 may include other similar instrumentalities for allowing computer programs or other instructions or data to be loaded into computing component 900. Such instrumentalities may include, for example, fixed or removable storage unit 940 and storage unit interface 935. Examples of such removable storage units 940 and storage unit interfaces 935 may include a program cartridge and cartridge interface, a removable memory (for example, a flash memory or other removable memory component) and memory slot, a PCMCIA slot and card, and other fixed or removable storage units 940 and storage unit interfaces 935 that allow software and data to be transferred from removable storage unit 940 to computing component 900.

Computing component 900 may also include a communications interface 950. Communications interface 950 may be used to allow software and data to be transferred between computing component 900 and external devices. Examples of communications interface 950 include a modem or softmodem, a network interface (such as an Ethernet, network interface card, WiMedia, IEEE 912.XX, or other interface), a communications port (such as for example, a USB port, IR port, RS232 port Bluetooth® interface, or other port), or other communications interface. Software and data transferred via communications interface 950 may typically be carried on signals, which may be electronic, electromagnetic (which includes optical) or other signals capable of being exchanged by a given communications interface 950. These signals may be provided to/from communications interface 950 via channel 945. Channel 945 may carry signals and may be implemented using a wired or wireless communication medium. Some non-limiting examples of channel 945 include a phone line, a cellular or other radio link, an RF link, an optical link, a network interface, a local or wide area network, and other wired or wireless communications channels.

In this document, the terms “computer program medium” and “computer usable medium” are used to generally refer to transitory or non-transitory media such as, for example, main memory 915, storage unit interface 935, removable storage media 925, and channel 945. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions may enable the computing component 900 or a processor to perform features or functions of the present application as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A method of improving attention, the method comprising:
presenting, via a display, first content to a user, wherein the first content comprises a first instruction and a first cue, and wherein the first instruction comprises information for the user to interact with the first cue, and wherein following the first set of instructions improves attention in the user;
tracking, with a movement tracking device, first user movement during presentation of the first content; and
evaluating, with a physical computer processor, the first user movement based on the first instruction to diagnose a first attention level in the user.

2. The method of claim 1, further comprising:
presenting, via the display, second content to the user, wherein the second content is dynamically adjusted based on an evaluation of the first user movement, and wherein the second content comprises a second instruction and a second cue;
tracking, with the movement tracking device, second user movement during presentation of the second content; and
evaluating, with the physical computer processor, the second user movement based on the second instruction to diagnose a second attention level in the user.

3. The method of claim 1, wherein the first user movement comprises movement of one of a user's eyes and a head.

4. The method of claim 1, wherein the first instruction comprises one of an instruction to focus the first user movement on the first cue, to focus the user movement away from the first cue, to focus the user movement on an opposite side of the first cue, and to use the first cue to direct future user movement.

5. The method of claim 1, wherein the first cue dynamically responds to the first user movement.

6. The method of claim 1, wherein tracking the first user movement comprises tracking an accuracy of the first user movement in relation to the first cue and tracking a timing of the first user movement in relation to the first cue.

7. The method of claim 1, wherein the first cue is presented for 50 milliseconds.

8. A method of improving autism spectrum disorder (ASD), the method comprising:
presenting, with a physical computer processor, first content to a user, wherein the first content comprises a first instruction and a first cue, and wherein the first instruction comprises information for the user to interact with the first cue, and wherein following the first set of instructions improves symptoms of ASD in the user;
tracking, with a movement tracking device, first user movement during presentation of the first content; and
evaluating, with the physical computer processor, the first user movement based on the first instruction to diagnose ASD in the user.

9. The method of claim 8, further comprising:
presenting, with the physical computer processor, second content to the user, wherein the second content is dynamically adjusted based on an evaluation of the first user movement, and wherein the second content comprises a second instruction and a second cue;
tracking, with the movement tracking device, second user movement during presentation of the second content; and
evaluating, with the physical computer processor, the second user movement based on the second instruction to determine changes to symptoms of the ASD in the user.

10. The method of claim 8, wherein the first user movement comprises movement of one of a user's eyes and a head.

11. The method of claim 8, wherein the first instruction comprises one of an instruction to focus on the first cue, look away from the first cue, look at an opposite side of the first cue, and use the first cue to direct future movement.

12. The method of claim 8, wherein the first cue dynamically responds to the first user movement by disappearing.

13. The method of claim 8, wherein tracking the first user movement comprises tracking an accuracy of the first user movement and tracking a timing of the first user movement.

14. The method of claim 8, wherein the first cue dynamically responds to the first user movement based on the first user movement being within less than a 2-inch by 2-inch square from a perspective of the user.

15. A method of improving attention deficit hyperactivity disorder (ADHD), the method comprising:
presenting, via a display, first content to a user, wherein the first content comprises a first instruction and a first cue, and wherein the first instruction comprises information for the user to interact with the first cue, and wherein following the first set of instructions improves symptoms of ADHD in the user;
tracking, with a movement tracking device, first user movement during presentation of the first content; and
evaluating, with a physical computer processor, the first user movement based on the first instruction to diagnose ADHD in the user.

16. The method of claim 15, further comprising:
presenting, via a display, second content to the user, wherein the second content is dynamically adjusted based on an evaluation of the first user movement, and wherein the second content comprises a second instruction and a second cue;
tracking, with the movement tracking device, second user movement during presentation of the second content; and
evaluating, with the physical computer processor, the second user movement based on the second instruction to determine changes to symptoms of the ADHD in the user.

17. The method of claim 15, wherein the first user movement comprises movement of one of a user's eyes and a head.

18. The method of claim 15, wherein the first instruction comprises one of an instruction to focus on the first cue, look away from the first cue, look at an opposite side of the first cue, and use the first cue to direct future movement.

19. The method of claim 15, wherein the first cue dynamically responds to the first user movement by providing audio.

20. The method of claim 15, wherein tracking the first user movement comprises tracking an accuracy of the first user movement and tracking a timing of the first user movement.
